(19) United States
(12) Patent Application Publication
(10) Pub. No.: US 2012/0076261 A1
(43) Pub. Date: Mar. 29, 2012

(54) RADIOLOGICAL IMAGE DISPLAYING APPARATUS AND METHOD

(75) Inventor: Takashi TAJIMA, Ashigarakami-gun (JP)

(73) Assignee: FUJIFILM CORPORATION, Tokyo (JP)

(21) Appl. No.: 13/247,087

(22) Filed: Sep. 28, 2011

(30) Foreign Application Priority Data
Sep. 29, 2010 (JP) 2010-219119

(51) Int. Cl. A61B 6/02

(52) U.S. Cl. 378/41

(57) ABSTRACT

Provided is a technique of making auxiliary lines easy to view when displaying a stereoscopic image using radiological images. Auxiliary lines are added on two radiological images for displaying a stereoscopic image, respectively. The auxiliary lines are made up of a plurality of grids appearing to be arranged in the depth direction, and, on the auxiliary lines that constitute a grid on a rear side, intersections with the auxiliary lines that constitute a grid on a front side and the near portion of the intersections are removed. In this way, the intersections of the grids can be viewed easily.

### BACKGROUND OF THE INVENTION

1. Field of the Invention

The present invention relates to a radiological image displaying apparatus and method for displaying stereoscopic images of a subject.

2. Description of the Related Art

In the related art, it is known that stereoscopic viewing can be realized using parallax by displaying a plurality of images in combination. Such an image (hereinafter referred to as a stereoscopic image or a 3D image) that can be viewed stereoscopically is displayed based on a plurality of images having parallaxes acquired by photographing the same subject from different directions.

Moreover, such way of generating stereoscopic image is utilized not only in the field of digital cameras and televisions but also in the field of radiography. That is, a technique which involves irradiating radioactive rays onto a subject from different radiographing directions, detecting radioactive rays having passed through the subject by a radiation detector to acquire a plurality of radiological images having parallaxes, and displaying a stereoscopic image using these radiological images has been performed. By using such a stereoscopic image, a user can observe radiological images having a sense of depth and more easily make a diagnosis.

However, since the radiological image is a transfer image of the inside of a subject, structures inside the subject, such as bones, various tissues and tumor masses, or lesions, such as calcification, are included in an overlapped state. Thus, when a stereoscopic image is displayed using radiological images, since structures are displayed stereoscopically with a stereoscopic effect so as to stay afloat in a space, it is difficult to figure out the sense of depth of structures in the stereoscopic image of radiological images. Moreover, a technique of using a 3D cursor capable of moving in the depth direction as well as in a planar direction to input necessary instructions on a displayed stereoscopic image may be considered. However, it is difficult to match the sense of depth of the 3D cursor so as to be identical to the sense of depth of a region of interest such as a lesion in a stereoscopic image. Thus, when displaying a stereoscopic image using a radiological image, a method of displaying auxiliary lines made up of a plurality of grids so as to be viewed stereoscopically may be considered.

However, when auxiliary lines are displayed so that a plurality of grids is arranged in the depth direction, since grids at different depths overlap each other as shown in FIG. 7, it is very difficult to observe the intersections of the grids at different depths. Thus, a technique of adding marks on the intersections of lines that constitute objects at the same depth when a plurality of objects is arranged in the depth direction is proposed in JP2003-515830A, for example. Moreover, a technique of adding marks on the intersections of meshes at the same depth is proposed in JP2009-015730A. Moreover, a technique of displaying a 3D mesh model so as to make lines on the rear side of the model invisible is also proposed in JP-2005-173685A and JP2004-297808A. Thus, when adding auxiliary lines on a stereoscopic image of radiological images, a technique of adding marks on the intersections of lines that constitute grids at the same depth and making lines of the grids on the rear side invisible can be considered.

### SUMMARY OF THE INVENTION

However, even when marks are added on the intersections of lines that constitute grids, since lines constituting a grid on the front side intersect lines constituting a grid on the rear side, it is not possible to eliminate the difficulty to observe the intersections of grids. Moreover, since the stereoscopic radiological image is a transfer image, if a grid on the rear side is made invisible, it is difficult to figure out the sense of depth of a stereoscopic image using auxiliary lines.

The present invention has been made in view of the above-mentioned problems and an object of the present invention is to provide a technique of making auxiliary lines easy to be viewed when displaying a stereoscopic image using radiological images.

According to an aspect of the present invention, a radiological image displaying apparatus includes: an image acquisition unit that acquires a plurality of radiological images for displaying a stereoscopic image of a subject; a display control unit that displays the stereoscopic image on a display unit using the plurality of radiological images; and an auxiliary line adding unit that adds auxiliary lines on the plurality of radiological images so as to be stereoscopically viewed in accordance with a sense of depth of the stereoscopic image, wherein the auxiliary lines are made up of a plurality of grids appearing to be arranged in the depth direction, and, on the auxiliary lines that constitute a grid on a rear side, intersections with the auxiliary lines that constitute a grid on a front side and the near portion of the intersections are removed.

Here, the rear side means a side facing the depth direction, and the front side means a side facing a direction opposite to the depth direction. Moreover, removing the portions near the intersections means removing the portions of the intersections to an extent such that the auxiliary lines constituting the grid on the front side and the auxiliary lines constituting the grid on the rear side appear to be discontinuous when the auxiliary lines are viewed stereoscopically.

The grid may have an optional shape such as a rectangular shape, a circular shape, or a triangular shape, and preferably, has a rectangular shape.

In the radiological image displaying apparatus of the above aspect of the present invention, the grids may be divided into a plurality of regions in a mesh shape.

In the radiological image displaying apparatus of the above aspect of the present invention, marks may be added on the intersections of the auxiliary lines constituting the grids.

According to another aspect of the present invention, a radiological image displaying method in a radiological image displaying apparatus, which includes an image acquisition unit that acquires a plurality of radiological images for displaying a stereoscopic image of a subject; and a display control unit that displays the stereoscopic image on a display unit using the plurality of radiological images, includes adding auxiliary lines on the plurality of radiological images so as to be stereoscopically viewed in accordance with a sense of depth of the stereoscopic image, wherein the auxiliary lines are made up of a plurality of grids appearing to be arranged in the depth direction, and intersections of auxiliary lines that constitute a grid on the front side and the auxiliary lines that constitute a grid on the rear side are removed; and portions of the auxiliary lines near the intersections are removed.

According to the above aspects of the present invention, the auxiliary lines which are made up of a plurality of grids appearing to be arranged in the depth direction are added on the plurality of radiological images so as to be stereoscopically viewed in accordance with a sense of depth of the stereoscopic image so that on the auxiliary lines that constitute a grid on a rear side, intersections with the auxiliary lines that constitute a grid on a front side and the near portion of the intersections are removed. Thus, since the intersections of the grids can be viewed more easily, grids which actually intersect each other and grids which do not intersect each other can be discriminated more easily when displaying a stereoscopic image using radiological images. As a result, the auxiliary lines can be made easy to view. Accordingly, by using the auxiliary lines when making diagnosis using stereoscopic images, a stereoscopic effect of the stereoscopic images can be figured out easily.

Moreover, by adding marks on the intersections of the auxiliary lines constituting the grids, grids which actually intersect each other and grids which do not intersect each other can be discriminated more easily.

### BRIEF DESCRIPTION OF THE DRAWINGS

FIG. 1 is a schematic configuration view of a radiological image radiographing apparatus to which a radiological image displaying apparatus according to an embodiment of the present invention is applied.

FIG. 2 is a view of an arm unit of the radiological image radiographing apparatus shown in FIG. 1 as viewed from the right side of FIG. 1.

FIG. 3 is a block diagram showing a simplified internal configuration of a computer of the radiological image radiographing apparatus shown in FIG. 1.

FIG. 4 is a view showing a radiological image on which auxiliary lines are added.

FIG. 5 is a flowchart showing processes performed in this embodiment.

FIG. 6 is a view showing another example of auxiliary lines.

FIG. 7 is a view showing auxiliary lines of the related art.

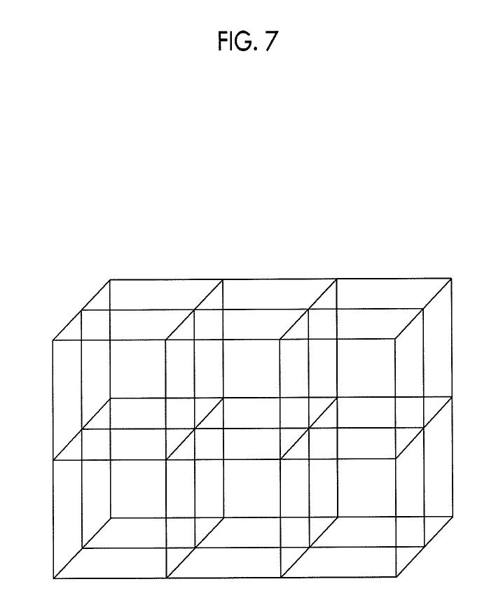

### DESCRIPTION OF THE PREFERRED EMBODIMENTS

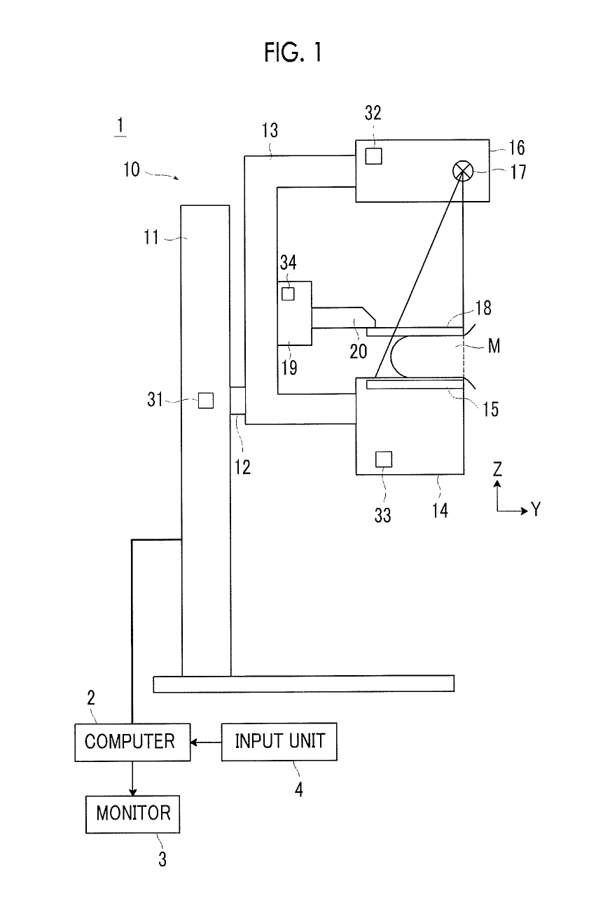

Hereinafter, an embodiment of the present invention will be described with reference to the accompanying drawings. FIG. 1 is a schematic configuration view of a radiological image radiographing apparatus to which a radiological image displaying apparatus according to an embodiment of the present invention is applied. A radiological image radiographing apparatus 1 according to this embodiment radiographs a breast M from different radiographing directions to acquire a plurality of radiological images in order to generate a stereoscopic image for stereoscopic viewing of a radiological breast image. As shown in FIG. 1, the radiological image radiographing apparatus 1 includes a radiographing unit 10 having an image acquisition unit (not shown), a computer 2 connected to the radiographing unit 10, a monitor 3 (display unit) connected to the computer 2, and an input unit 4.

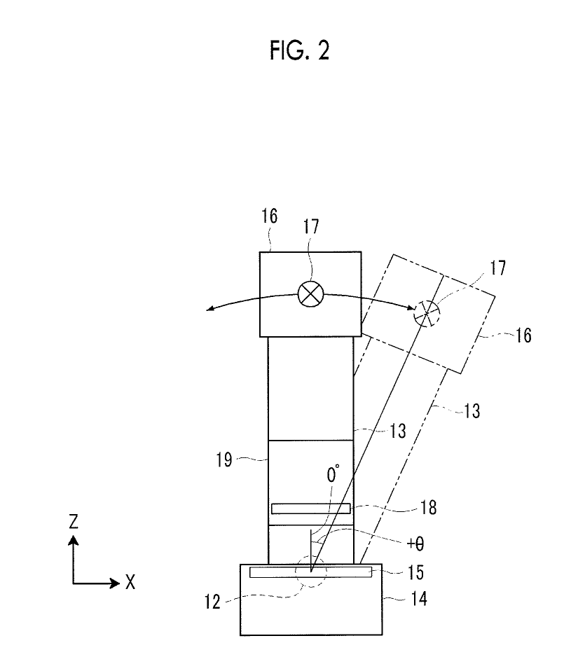

The radiographing unit 10 includes a base 11, a rotation shaft 12 that is movable in the vertical direction (Z direction) and rotatable with respect to the base 11, and an arm unit 13 that is connected to the base 11 by the rotation shaft 12. FIG. 2 shows the arm unit 13 as viewed from the right side of FIG. 1.

The arm unit 13 has a C-shape and includes one end to which a radiography platform 14 is attached and the other end to which a radiation irradiation unit 16 is attached so as to face the radiography platform 14. The rotation and vertical movement of the arm unit 13 are controlled by an arm controller 31 that is incorporated into the base 11.

The radiography platform 14 includes a radiation detector 15 such as a flat panel detector, and a detector controller 33 that controls the reading of a charge signal from the radiation detector 15.

Moreover, the radiography platform 14 includes, for example, a circuit board on which a charge amplifier that converts the charge signal read from the radiation detector 15 into a charge signal, a correlated double sampling circuit that samples the charge signal output from the charge amplifier, and an A/D converter that converts the charge signal into a digital signal are formed.

Moreover, the radiography platform 14 is configured so as to be rotatable with respect to the arm unit 13. Therefore, even when the arm unit 13 is rotated with respect to the base 11, the direction of the radiography platform 14 can be fixed with respect to the base 11.

The radiation detector 15 can repeatedly record and read the radiological image and may be a so-called direct-conversion radiological image detector that directly receives radiation to generate charge or a so-called indirect-conversion radiological image detector that converts radiation into visible light and then converts the visible light into a charge signal. As a method of reading a radiological image signal, it is preferable to use a so-called TFT (thin film transistor) reading method of turning on or off a TFT switch to read the radiological image signal or a so-called optical reading method of emitting reading light to read the radiological image signal. However, the reading method is not limited thereto, and other methods may be used.

The radiation irradiation unit 16 includes a radiation source 17 and a radiation source controller 32. The radiation source controller 32 controls the time when radiation is emitted from the radiation source 17 and the radiation generation conditions (for example, a tube current, time, and a tube current-time product) of the radiation source 17.

In addition, a compression plate 18 that is provided above the radiography platform 14 and compresses the breast, a supporting portion 20 that supports the compression plate 18, and a moving mechanism 19 that moves the supporting portion 20 in the vertical direction (Z direction) are provided at the center of the arm unit 13. The position and compression pressure of the compression plate 18 are controlled by a compression plate controller 34.

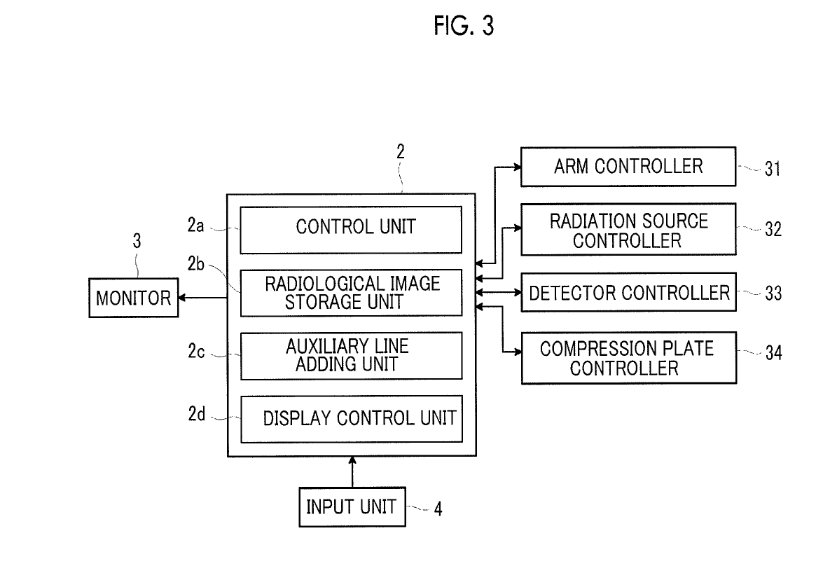

The computer 2 includes, for example, a central processing unit (CPU) and a storage device, such as a semiconductor memory, a hard disk, or an SSD (Solid State Drive). A control unit 2a, a radiological image storage unit 2b, an auxiliary line adding unit 2c (auxiliary line adding unit), and a display control unit 2d (display control unit) shown in FIG. 3 are formed by these hardware components.

The control unit 2a outputs predetermined control signals to various kinds of controllers 31 to 34 to control the entire system.

The radiological image storage unit 2b stores two radiological images (G1 and G2) detected by the radiation detector 15 by radiographing from two different radiographing directions.

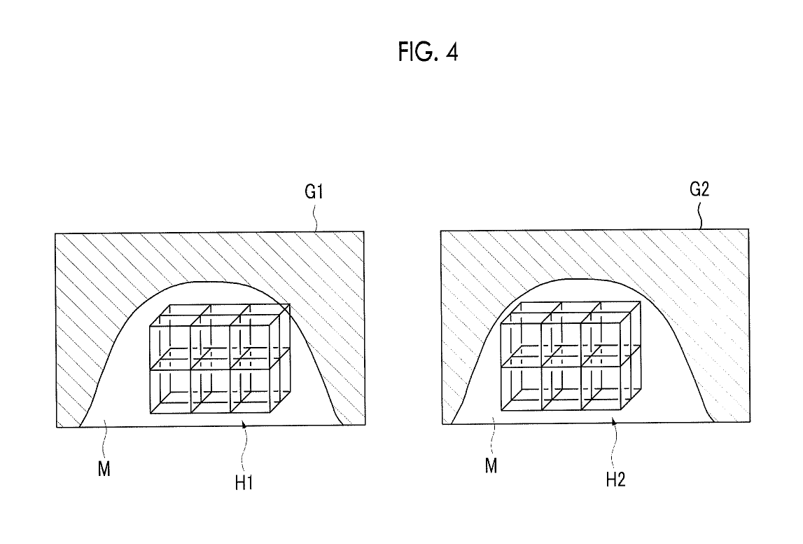

The auxiliary line adding unit 2c adds auxiliary lines for expressing the sense of depth of a stereoscopic image on the two radiological images G1 and G2 so as to be viewed stereoscopically when a stereoscopic image using the two radiological images G1 and G2 is displayed on the monitor 3. FIG. 4 is a view showing two radiological images on which auxiliary lines are added. As shown in FIG. 4, auxiliary lines H1 and H2 made up of a plurality of grids are added on the radiological images G1 and G2, respectively, so as to be superimposed on the breast M in the radiological images G1 and G2. The auxiliary lines H1 and H2 are made up of three rectangular grids, and the respective grids are divided into a plurality of regions in a mesh shape. Moreover, the intersections of lines that constitute a grid on the rear side and lines that constitute a grid on the front side are removed, and portions of the lines near the intersections are removed. In FIG. 4, the auxiliary lines between grids extending in the depth direction are not essential configurations but are provided for better understanding of the present invention. However, by providing the auxiliary lines in the depth direction, the auxiliary lines are made easier to be viewed, and a more effective stereoscopic effect can be obtained.

Moreover, the parallaxes of the respective grids may be determined to be evenly divided, for example, based on the largest parallax and the smallest parallax between corresponding structures included in the radiological images G1 and G2. When a stereoscopic image is displayed using the radiological images G1 and G2 on which the auxiliary lines H1 and H2 are added, the parallaxes of the auxiliary lines H1 and H2 may be changed in accordance with the input from the input unit 4. By doing so, the stereoscopic effect of the auxiliary lines can be made identical to the stereoscopic effect of the breast M included in the stereoscopic image. Thus, auxiliary lines can be added on a plurality of radiological images so as to be stereoscopically viewed in accordance with the sense of depth of the stereoscopic image. Moreover, whether or not to display the auxiliary lines H1 and H2 may be switched in accordance with the input from the input unit 4. Furthermore, since the region of the breast M in the radiological images G1 and G2 has a relatively high luminance, the auxiliary lines H1 and H2 are preferably displayed in a low luminance.

The display control unit 2d performs predetermined processing on the radiological images G1 and G2 on which the auxiliary lines H1 and H2 are added and then displays the stereoscopic image of the breast M on the monitor 3.

The monitor 3 is configured to be able to display a stereoscopic image in 3D using the two radiological images G1 and G2 output from the computer 2. As an example of a 3D display method used in the monitor 3, a method in which two radiological images are displayed using two image planes so that the right eye of a viewer sees one radiological image and the left eye of the viewer sees the other radiological image using a half mirror, polarized glasses, or the like, to thereby display a stereoscopic image can be adopted. Moreover, a method in which two radiological images are superimposed on each other and observed by a viewer wearing polarized glasses, to thereby display a stereoscopic image may be used. Furthermore, a method like a parallax barrier method and a lenticular method, in which the monitor 3 is configured by a 3D liquid crystal display so that two radiological images can be viewed stereoscopically, may be used.

The input unit 4 includes a keyboard or a pointing device, such as a mouse, and receives an input of radiographing conditions, an input of a radiographing start instruction, and the like, from a radiographer.

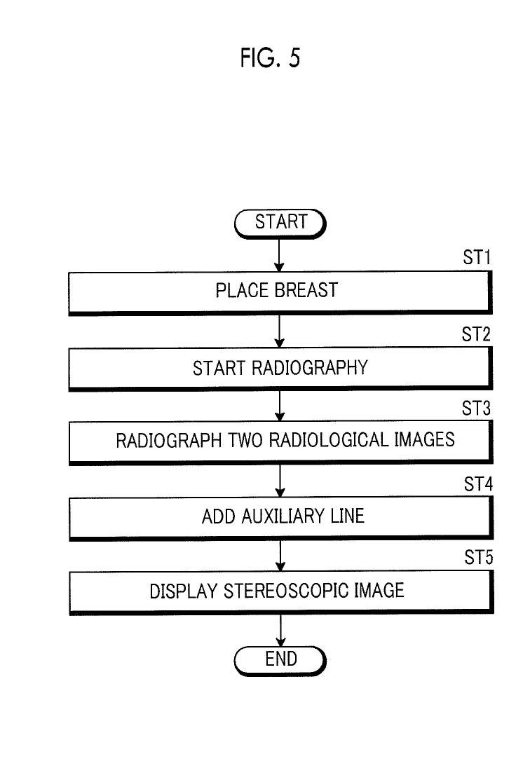

Next, processes performed in this embodiment will be described. FIG. 5 is a flowchart showing processes performed in this embodiment. First, the breast M of a patient is placed on the radiography platform 14 and the compression plate 18 compresses the breast M with a predetermined pressure (step ST1). Subsequently, the input unit 4 sequentially receives various kinds of radiographing conditions and a radiographing start instruction (step ST2).

When the input unit 4 receives the radiographing start instruction, two radiological images for displaying a stereoscopic image of the breast M are radiographed (step ST3). Specifically, first, the control unit 2a reads the angle of convergence κ stored therein and outputs the information of the read angle of convergence κ to the arm controller 31. The arm controller 31 receives the information of the angle of convergence κ output from the control unit 2a. Then, the arm controller 31 first outputs a control signal so as to move the arm unit 13 to be arranged in a direction (the direction of 0°) vertical to the radiography platform 14 as indicated by the solid line in FIG. 2.

In a state where the arm unit 13 is moved to be vertical to the radiography platform 14 in accordance with the control signal output from the arm controller 31, the control unit 2a outputs control signals to the radiation source controller 32 and the detector controller 33 so as to irradiate radioactive rays and read radiological image signals, respectively. The position of the radiation source 17 in this state corresponds to the reference viewpoint position. In response to the control signals, the radiation source 17 irradiates radioactive rays, the radiation detector 15 detects a radiological image of the breast M radiographed from the direction of 0°, and the detector controller 33 reads a radiological image signal from the radiation detector 15. Then, predetermined signal processing is performed on the radiological image signal, and the radiological image signal is stored in the radiological image storage unit 2b of the computer 2 as a reference radiological image G1.

Subsequently, the arm controller 31 outputs a control signal so as to rotate the arm unit 13 by +θ in the direction vertical to the radiography platform 14 as indicated by an imaginary line in FIG. 2. Moreover, in a state where the arm unit 13 is rotated by +θ in accordance with the control signal output from the arm controller 31, the control unit 2a outputs control signals to the radiation source controller 32 and the detector controller 33 so as to irradiate radioactive rays and read radiological image signals, respectively. In response to the control signals, the radiation source 17 irradiates radioactive rays, the radiation detector 15 detects a radiological image of the breast M radiographed from the direction of +θ°, and the detector controller 33 reads a radiological image signal. Then, the radiological image signal is subjected to predetermined signal processing and is then stored in the radiological image storage unit 2b of the computer 2 as a radiological image G2.

Then, the two radiological images G1 and G2 stored in the radiological image storage unit 2b are read, and the auxiliary line adding unit 2c adds the auxiliary lines H1 and H2 on these radiological images G1 and G2, respectively (step ST4). Moreover, the display control unit 2d performs predetermined processing on radiological images GS1 and GS2 on which the auxiliary lines H1 and H2 are added, and the processed radiological images are output to the monitor 3, whereby a stereoscopic image of the breast M is displayed on the monitor 3 (step ST5).

As above, in this embodiment, the auxiliary lines H1 and H2 which are made up of a plurality of grids appearing to be arranged in the depth direction are added on the radiological images G1 and G2 so as to be stereoscopically viewed in accordance with a sense of depth of the stereoscopic image so that the intersections of lines that constitute a grid on the front side and lines that constitute a grid on the rear side are removed, and portions of the lines near the intersections are removed. Thus, since the intersections of the grids can be viewed more easily, grids which actually intersect each other and grids which do not intersect each other can be discriminated more easily when displaying a stereoscopic image using the radiological images G1 and G2. As a result, the auxiliary lines H1 and H2 can be made easy to view. Accordingly, by using the auxiliary lines H1 and H2 when making diagnosis using stereoscopic images, a stereoscopic effect of the stereoscopic images can be figured out easily.

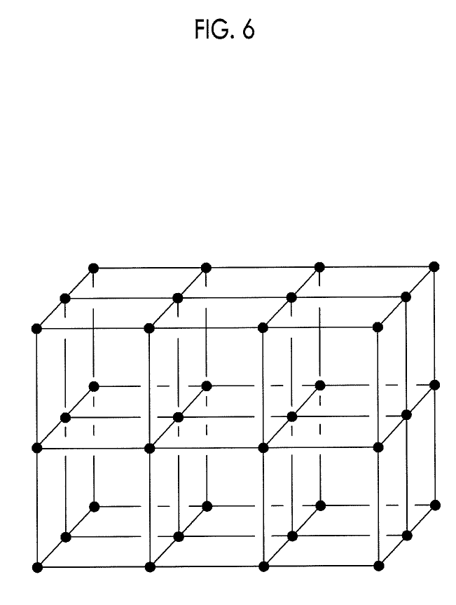

In the above embodiment, marks such as black circular dots as shown in FIG. 6 may be added to the intersections of lines constituting grids at respective depths. In this way, grids which actually intersect each other and grids which do not intersect each other can be discriminated more easily.

Moreover, in the above embodiment, although auxiliary lines made up of three grids arranged in the depth direction are displayed, an optional number of grids may be used in accordance with the stereoscopic effect of the stereoscopic image if the number of grids is 2 or more. Moreover, the number of mesh-shaped divided regions in the grid is not limited to that shown in FIG. 4, and an optional number of divided regions may be used.

Furthermore, in the above embodiment, although a rectangular grid is used, a grid having an optional shape such as a circular shape or a triangular shape may be used.

Furthermore, in the above embodiment, although a radiological image acquired by radiographing the breast M from the direction of 0° is used as the reference radiological image G1, a radiological image acquired by radiographing the breast M from a direction different from the direction of 0° may be used as the reference radiological image of the two radiological images for displaying a stereoscopic image. In this case, a stereoscopic image may be displayed using the radiological image radiographed from the direction different from the direction of 0° as the reference radiography G1.

Furthermore, in the above embodiment, although the radiological image radiographing apparatus to which the radiological image displaying apparatus of the present invention is applied has been described to be an apparatus for radiographing a radiological breast image, the subject of the present invention is not limited to a breast. For example, a radiological image radiographing apparatus which radiographs the chest, the head, and the like may be used.

What is claimed is:

1. A radiological image displaying apparatus comprising:
an image acquisition unit that acquires a plurality of radiological images for displaying a stereoscopic image of a subject;
a display control unit that displays the stereoscopic image on a display unit using the plurality of radiological images; and
an auxiliary line adding unit that adds auxiliary lines on the plurality of radiological images so as to be stereoscopically viewed in accordance with a sense of depth of the stereoscopic image,
wherein the auxiliary lines are made up of a plurality of grids appearing to be arranged in a depth direction, and, on the auxiliary lines that constitute a grid on a rear side, intersections with the auxiliary lines that constitute a grid on a front side and the near portion of the intersections are removed.

2. The radiological image displaying apparatus according to claim 1,
wherein the grids are divided into a plurality of regions in a mesh shape.

3. The radiological image displaying apparatus according to claim 1,
wherein marks are added on the intersections of the lines constituting the grids.

4. The radiological image displaying apparatus according to claim 2,
wherein marks are added on the intersections of the lines constituting the grids.

5. A radiological image displaying method in a radiological image displaying apparatus including:
an image acquisition unit that acquires a plurality of radiological images for displaying a stereoscopic image of a subject; and
a display control unit that displays the stereoscopic image on a display unit using the plurality of radiological images, the method comprising:
adding auxiliary lines on the plurality of radiological images so as to be stereoscopically viewed in accordance with a sense of depth of the stereoscopic image,
wherein the auxiliary lines are made up of a plurality of grids appearing to be arranged in a depth direction, and, on the auxiliary lines that constitute a grid on a front side, the intersections with the auxiliary lines that constitute a grid on a front side and the near portion of the intersections are removed.
